(19) United States
(12) Patent Application Publication
(10) Pub. No.: US 2024/0352491 A1
(43) Pub. Date: Oct. 24, 2024

(54) CO2-FREE WASTE GAS FERMENTATION USING ACETOGEN STRAINS AND ACETIC ACID PRODUCTION METHOD ACCORDINGLY

(71) Applicant: GWANGJU INSTITUTE OF SCIENCE AND TECHNOLOGY, Gwangju (KR)

(72) Inventors: In Seop CHANG, Gwangju (KR); Ji Yeon KIM, Gwangju (KR); Mun Gyu LEE, Gwangju (KR)

(21) Appl. No.: 18/639,266

(22) Filed: Apr. 18, 2024

(30) Foreign Application Priority Data
Apr. 20, 2023 (KR) 10-2023-0051824

(51) Int. Cl. C12P 7/54; C12M 1/00

(52) U.S. Cl. CPC C12P 7/54; C12M 29/08; C12M 29/18; C12M 47/10

(57) ABSTRACT

Proposed is a method for CO₂-free waste gas fermentation using microbial strains, and the method is capable of maximizing a consumption rate of gaseous substrates by additionally injecting methanol during a fermentation process of the microbial strains, and converting CO₂, a greenhouse gas, generated during the fermentation process into hydrocarbon products therethrough. The method may comprise: 1) injecting microbial strains into a bioreactor; and 2) injecting gaseous substrates and methanol into the bioreactor, and converting the gaseous substrates and the methanol into hydrocarbon products through fermentation of the microbial strains, wherein all CO₂ present inside the bioreactor is consumed and converted into hydrocarbon products.

### TECHNICAL FIELD

The present disclosure relates to a method for CO₂-free waste gas fermentation using microbial strains.

### BACKGROUND ART

As problems such as environmental problems and energy exhaustion caused by the use of petroleum-based compounds become more serious, the world is reducing carbon dioxide emission to prevent global warming caused by the use of fossil fuels, while accelerating development of various alternative energy sources in preparation for high oil prices.

Syngas, a representative alternative energy source, may be produced through a process of reforming natural gas, or gasifying solid raw materials such as coal, organic waste and biomass.

Such syngas has the following advantages as an alternative energy source.

First, syngas may be converted from most hydrocarbons, thereby having a low risk of raw material exhaustion, and the raw materials may be stably secured since, compared to fossil fuels, price fluctuations are low.

Second, syngas-based energy conversion has a form of using carbon dioxide instead of releasing carbon dioxide, and therefore, concern about environmental pollution caused by carbon dioxide emission is small.

Third, since the main components are hydrogen and carbon, syngas may be converted to various high value-added products such as acetic acid, butyric acid, ethanol and butanol, making it highly usable and economical.

Such syngas may be used through a biorefinery process using microorganisms. Typically, anaerobic acetate producing bacteria commonly known as acetogen are used. Acetogen fixes C₁ gas such as carbon monoxide or carbon dioxide to acetyl-CoA through the Wood-Ljungdahl pathway, and, by converting it to an organic acid such as acetic acid, energy required for cell growth dynamics is obtained.

A syngas biorefinery process using acetogen is directly affected by microorganism growth rate and gas consumption rate, and therefore, has problems of relatively low reaction rate and production efficiency compared to existing chemical conversion processes.

In particular, unlike traditional fermentation technology based on dissolved organic substances such as glucose and fructose, operating a bioprocess using syngas, a gaseous substrate, has had problems of even lower microorganism growth and metabolism rates since the syngas has very low solubility in an aqueous phase, a culture condition of microorganisms.

Accordingly, studies to increase process performance through enhancing efficiency of a biorefinery process, and furthermore, to develop an economical process system have been actively ongoing.

### PRIOR ART DOCUMENTS

### Patent Documents

(Patent Document 1) KR 10-2014-0026207 A

### DISCLOSURE

### Technical Problem

The present disclosure is directed to providing a method for CO₂-free waste gas fermentation using microbial strains.

The present disclosure is also directed to providing a method for CO₂-free waste gas fermentation using microbial strains, the method capable of maximizing a consumption rate of gaseous substrates by additionally injecting methanol during a fermentation process of the microbial strains, and converting all CO₂, a greenhouse gas, generated during the fermentation process into hydrocarbon products therethrough.

The present disclosure is also directed to providing a system for CO₂-free waste gas fermentation using microbial strains.

### Technical Solution

In view of the above, a method for CO₂-free waste gas fermentation using microbial strains according to one embodiment of the present disclosure includes 1) injecting microbial strains into a bioreactor; and 2) injecting gaseous substrates and methanol into the bioreactor, and converting the gaseous substrates and the methanol into hydrocarbon products through fermentation of the microbial strains, wherein all CO₂ present inside the bioreactor is consumed and converted into hydrocarbon products.

The gaseous substrate includes one or more gases selected from the group consisting of CO, H₂ and CO₂.

The hydrocarbon product is selected from the group consisting of acetate, butyrate, ethanol, propanol, butanol, 2,3-butanediol, propionate, caproate, propylene, butadiene, isobutylene, ethylene and mixtures thereof.

The microbial strain is an acetogen strain, and the acetogen strain is one or more strains selected from the group consisting of Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, Moorella thermoacetica and Eubacterium callanderi.

The methanol is capable of increasing a consumption rate of the gaseous substrates by the microbial strains.

An injection rate (vvm) of the gaseous substrates into the bioreactor is from 0.005 vvm to 0.010 vvm.

A pH condition inside the bioreactor is from 6.8 to 7.2.

A hydrocarbon product according to another embodiment of the present disclosure is produced using the above-described fermentation method.

A system for CO₂-free waste gas fermentation using microbial strains according to another embodiment of the present disclosure includes a bioreactor including microbial strains; a gas cylinder capable of injecting gaseous substrates into the bioreactor; and a liquid inlet capable of injecting methanol into the bioreactor, wherein the gaseous substrates and the methanol are injected into the bioreactor, and the gaseous substrates and the methanol are converted into hydrocarbon products through fermentation of the microbial strains.

The system for CO₂-free waste gas fermentation is capable of consuming and converting all CO₂ present inside the bioreactor into hydrocarbon products.

The system for CO₂-free waste gas fermentation is capable of independently controlling one or more variables selected from the group consisting of an injection rate of the gaseous substrates, a pH inside the reactor and a mixing ratio of the gaseous substrates.

### Advantageous Effects

The present disclosure relates to a method for CO₂-free waste gas fermentation using microbial strains, and is effective in maximizing a consumption rate of gaseous substrates by additionally injecting methanol during a fermentation process of the microbial strains, and converting all CO₂, a greenhouse gas, generated during the fermentation process into hydrocarbon products therethrough.

### BRIEF DESCRIPTION OF DRAWINGS

FIG. 1 illustrates a schematic diagram of a system for CO₂-free waste gas fermentation using microbial strains according to one embodiment of the present disclosure.

FIG. 2 shows results of analyzing genome sequences of seven methylotrophic acetogen strains according to one embodiment of the present disclosure.

FIG. 3 illustrates a two-phase reaction mode of a CO consumed stage and a CO₂ consumed stage of a method for waste gas fermentation according to one embodiment of the present disclosure.

FIG. 4 shows a reducing power and a CO₂ consumption path under a H₂/CO₂ (8:2) and methanol (30 mM)/CO₂ condition according to one embodiment of the present disclosure.

FIG. 5 shows results of monitoring gaseous substrate consumption depending on the presence or absence of methanol supply during syngas fermentation according to one embodiment of the present disclosure.

FIG. 6 shows a process operation profile through a bioreactor designed according to one embodiment of the present disclosure.

FIG. 7 shows results of monitoring changes in gaseous substrates and hydrocarbon products when driving a bioreactor according to one embodiment of the present disclosure.

FIG. 8 shows a total carbon content of gaseous substrates, hydrocarbon products and biomass for each stage of stage 1 to stage 4 in a method for waste gas fermentation according to one embodiment of the present disclosure.

FIG. 9 shows a prediction of cumulative CO₂ emission inside a bioreactor under various reaction conditions in a method for waste gas fermentation according to one embodiment of the present disclosure.

### BEST MODE

Hereinafter, embodiments of the present disclosure will be described in detail so that those skilled in the art may readily implement the present disclosure. However, the present disclosure may be embodied in various different forms, and is not limited to the embodiments described herein.

Thermochemical processes that gasify various feedstocks including biomass, coal and municipal solid waste convert these resources into usable energy mostly having a form of syngas in which carbon monoxide (CO), hydrogen (H₂) and carbon dioxide (CO₂) are mixed. Traditionally, some of these syngas components have been used to produce thermal energy and power using their flammable properties, however, there has been a problem in that the accompanying CO₂ emission accounts for a significant portion of greenhouse gas in the atmosphere causing man-made climate changes. Since a gas is a very small molecule and generally coexists with other gas compounds, purifying a single gas for a specific purpose is very difficult, and in terms of this, a system capable of completely converting or upcycling a syngas mixture into a value-added material without gas separation and purification is very important.

Therefore, biological conversion by microorganisms through syngas fermentation will be a very useful means in that biocatalytic microorganisms such as acetogen strains are naturally equipped with physiological tools absorbing CO, CO₂ and H₂ as an energy source.

However, such a strategy of utilizing syngas through microbial strains has limitations in actual applications due to problems of reliability and availability. What is important is that an end product of syngas fermentation includes not only beneficial chemicals, but also CO₂, a greenhouse gas, as described above.

Accordingly, it is necessary to develop a syngas fermentation process capable of minimizing the amount of residual CO₂ generated during a syngas fermentation process using microbial strains, and completely converting the syngas to liquid chemicals that are beneficial chemicals.

Meanwhile, considering that methanol may be included in an intermediate of a microbial syngas utilization pathway and may be metabolized into acetate and butyrate when combined with CO₂ afterward, the inventors of the present disclosure intended to use methanol as an auxiliary substrate capable of facilitating efficiency of syngas fermentation by microorganisms.

Accordingly, as a result of identifying the effect of methanol on syngas fermentation using Eubacterium limosum, a methylotrophic acetogen strain, the inventors have identified that selective supply of additional H₂ and methanol during a fermentation process consumes all residual CO₂, and is ultimately capable of completely converting the CO₂ into butyrate and acetate that are value-added materials.

Therefore, the present disclosure is directed to providing a novel process capable of accelerating an overall gaseous substrate consumption rate of a syngas fermentation process using microorganisms by utilizing H₂ and/or methanol as a “core controller”, and more specifically, converting all CO₂ generated during the fermentation process into hydrocarbon products such as butyrate and acetate without additional separation and purification processes.

In view of the above, a method for CO₂-free waste gas fermentation using microbial strains according to one embodiment of the present disclosure includes 1) injecting microbial strains into a bioreactor; and 2) injecting gaseous substrates and methanol into the bioreactor, and converting the gaseous substrates and the methanol into hydrocarbon products through fermentation of the microbial strains, and the method is capable of consuming and converting all CO₂ present in the bioreactor into hydrocarbon products.

Specifically, the present disclosure relates to a fermentation process converting both gaseous substrates and methanol into hydrocarbon products through a metabolic action of microbial strains present inside a bioreactor.

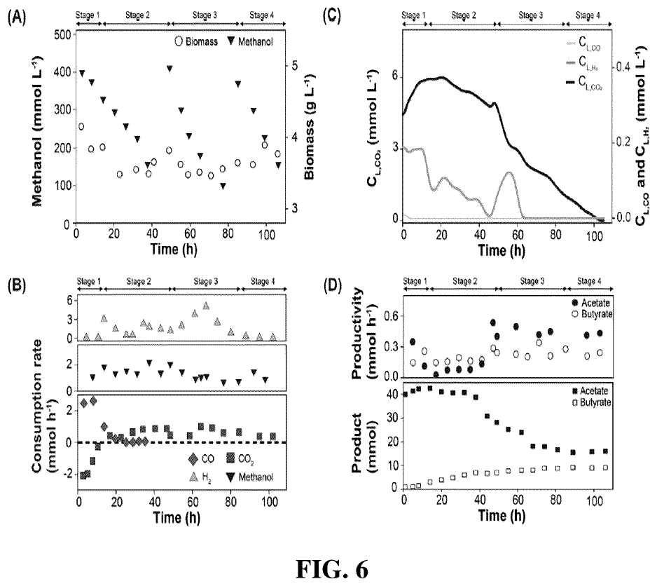

Meanwhile, when the step 2) is time sequentially divided, it may be divided into a CO consumed stage (stage 1 of FIGS. 6 to 8) and H₂, CO₂ and methanol (CH₃OH) consumed stages (stages 2 to 4 of FIGS. 6 to 8), and the initial CO consumed stage is a stage of consuming CO to produce CO₂. Meanwhile, when all the CO is consumed, the stages of simultaneously consuming H₂, CO₂ and methanol (CH₃OH) proceed. In other words, in the present disclosure, CO₂ is both a gaseous substrate and a product.

In other words, the stages proceed such that CO is all consumed through the stage of consuming all CO among the gaseous substrates at the beginning of the fermentation process of the present disclosure, and then H₂ and CO₂ that are the remaining gaseous substrates, and liquid methanol are consumed simultaneously, and as a result, the gaseous substrates and the methanol may be completely converted into hydrocarbon products.

Meanwhile, the core of the fermentation process of the present disclosure lies in mixing and injecting methanol together with the gaseous substrates. Specifically, the methanol may maximize a consumption rate of H₂ and CO₂ during the metabolic process of microbial strains, and through this, all the gaseous substrates may be converted into hydrocarbon products in the fermentation process without emission of residual CO₂.

The gaseous substrate includes one or more gases selected from the group consisting of CO, H₂ and CO₂.

The term “gaseous substrate” in the present specification may include a waste gas itself including one or more gases selected from the group consisting of CO, H₂ and CO₂, or one or more gases selected from the group consisting of CO, H₂ and CO₂ separated and purified from the waste gas.

Meanwhile, the “waste gas” may be a waste gas obtained as a by-product of an industrial process or from some other supply sources such as automobile exhaust fumes or biomass gasification.

Specifically, the “industrial process” may be selected from the group consisting of ferrous metal product manufacturing such as steel manufacturing, non-ferrous metal product manufacturing, petroleum refining, coal gasification, electric power production, carbon black production, ammonia production, methanol production and coke manufacturing.

Specifically, the gaseous substrate may include CO, H₂ and CO₂, may include CO, H₂ and CO₂ in a partial pressure ratio of 1:1:1 to 1:3:2 and more specifically in a partial pressure ratio of 1:1:1 to 1:2.5:1.5, and preferably includes CO, H₂ and CO₂ in a partial pressure ratio of 22:50:28. When the partial pressure ratio of the gaseous substrates injected into the bioreactor is in the above-mentioned range, metabolic efficiency of the microbial strains inside the bioreactor may be maximized, which has an advantage of increasing the consumption rate of the gaseous substrates.

However, when the weight is greater than or less than the above-mentioned weight range, a balance between each gas consumed during the metabolic process of the microbial strains is broken, which may reduce metabolic efficiency of the microbial strains.

The hydrocarbon product is selected from the group consisting of acetate, butyrate, ethanol, propanol, butanol, 2,3-butanediol, propionate, caproate, propylene, butadiene, isobutylene, ethylene and mixtures thereof.

More Specifically, the hydrocarbon product is selected from the group consisting of acetate, butyrate and mixtures thereof.

The microbial strain is an acetogen strain, and the acetogen strain is one or more strains selected from the group consisting of Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, Moorella thermoacetica and Eubacterium callanderi.

However, the microbial strain is not limited to the above-exemplified microorganism types, and is, as a strain capable of using the gaseous substrates and the strains of the present disclosure in a metabolic process, defined to include all microorganism types capable of converting the gaseous substrates and the methanol into the hydrocarbon products of the present disclosure, a fermentation product.

Specifically, the microbial strain of the present disclosure is preferably Eubacterium limosum or Eubacterium callanderi, and is more preferably Eubacterium limosum KIST612 or Eubacterium callanderi KIST612 strain.

The Eubacterium limosum KIST612 or Eubacterium callanderi KIST612 strain is an acetogen strain capable of methanol methylotrophy, and is capable of metabolizing methanol by methanol dehydrogenase or methyltransferase, and some of them use a methyl transfer system to use methanol. In addition, it includes an operon (mta operon) encoding methyltransferase similar to Acetobacterium woodie, a closely related acetogen strain. Accordingly, the Eubacterium limosum KIST612 strain of the present disclosure is capable of increasing autotrophic metabolism of the strain by converting methanol into methyl-THF, an intermediate metabolite of Wood-Ljungdahl pathway, using the methyltransferase, and ultimately, is capable of increasing a consumption rate of the gaseous substrates by the acetogen strain, and increasing efficiency of the biorefinery process.

The methanol is capable of increasing a consumption rate of the gaseous substrates by the microbial strain.

Specifically, as described above, the microbial strain of the present disclosure uses gaseous substrates for metabolism, thereby converting the gaseous substrates into the hydrocarbon products of the present disclosure.

However, such a biorefinery process of syngas using microbial strains is directly affected by a growth rate of microbial strains and a consumption rate of the gaseous substrates, and therefore, has problems of not only having relatively low reaction rate and production efficiency compared to existing chemical conversion processes, but also reducing growth and metabolic rates of the microbial strains particularly when using the syngas as a raw material due to low solubility of the syngas components.

Accordingly, by additionally injecting the methanol, which is capable of being converted into an intermediate product of Wood-Ljungdahl pathway in which the acetogen strain fixes gaseous substrates such as CO and CO₂ to acetyl-CoA, in the fermentation process of the microbial strains, the inventors of the present disclosure are able to increase the concentration of the metabolic intermediate product, which ultimately leads to an effect of increasing overall metabolic efficiency.

In other words, when additionally injecting methanol in the syngas fermentation process of the present disclosure, the consumption rate of the gaseous substrates by the microbial strains may increase, and as a result, the amount of residual CO₂ in the microbial metabolic process targeted in the present disclosure is converged to “0 (zero)”. Preferably, the methanol is capable of increasing the consumption rates of H₂ and CO₂ among the gaseous substrates.

Meanwhile, in the method for waste gas fermentation of the present disclosure, the step 2) is a step of injecting gaseous substrates and methanol into the bioreactor, and, by driving the bioreactor, converting the gaseous substrates and the methanol into hydrocarbon products through fermentation of the microbial strains, and, in order to prevent all of hydrogen (H₂) and methanol (CH₃OH) present inside the reactor from being consumed, may preferably include additionally injecting hydrogen (H₂) and methanol (CH₃OH) into the reactor at a specific point. More specifically, the step 2) may include 2-1) additionally injecting hydrogen (H₂) and methanol (CH₃OH) into the bioreactor at a point when the amounts of hydrogen (H₂) and methanol (CH₃OH) inside the reactor become 0.01 mM or less and 100 mM or less, respectively, after performing the step 2. Through this, the consumption of CO₂ remaining inside the bioreactor may increase.

Meanwhile, in another aspect, the step 2) may further include 2-1) additionally injecting H₂ and methanol into the bioreactor at a point when 40 hours to 60 hours pass after performing the step 2), and through this, the consumption of CO₂ remaining inside the bioreactor may increase.

The additionally injected hydrogen (H₂) and methanol (CH₃OH) may be injected through a gas cylinder and a liquid inlet, respectively. More specifically, 80 mM to 100 mM of hydrogen (H₂) and 350 mM to 400 mM of methanol (CH₃OH) are injected at a point when the amounts of hydrogen (H₂) and methanol (CH₃OH) become 0.01 mM or less and 100 mM or less, respectively, or at a point when 40 hours to 50 hours pass after performing the step 2). When injected in the above-mentioned range, consumption efficiency of the gaseous substrates targeted in the present disclosure may be maximized.

More preferably, the step 2) may further include 2-2) additionally injecting methanol into the bioreactor at a point when 80 hours to 100 hours pass after performing the step 2), and this may allow the gaseous substrates present inside the bioreactor, particularly the residual CO₂, to be all consumed. In other words, the methanol additionally injected in the step 2-2) is to convert all CO₂ remaining inside the bioreactor into hydrocarbon products, and through this, all CO₂, a greenhouse gas, generated in the fermentation process is converted into hydrocarbon products, and as a result, a syngas fermentation process with no CO₂ emission may be provided.

The additionally injected methanol may be injected through a liquid inlet. More specifically, the methanol is preferably injected at a concentration of 350 mM to 400 mM at a point when 80 hours to 90 hours pass after performing the step 2), and when injected in the above-mentioned range, consumption efficiency of the residual CO₂ may be maximized.

The injection rate (vvm) of the gaseous substrates into the bioreactor is from 0.005 vvm to 0.010 vvm.

The gaseous substrate injection rate (vvm, volume per volume per minute) represents an injection rate of the gaseous substrate of the present disclosure into the reactor, and the injection rate (vvm) of the gaseous substrates into the bioreactor is from 0.005 vvm to 0.010 vvm, and more preferably 0.008 vvm.

When the gaseous substrates are injected in the above-mentioned injection rate range, the effect of gaseous substrate consumption by the microbial strains may be maximized inside the bioreactor.

A pH condition inside the bioreactor is from 6.8 to 7.2, and is preferably 7.0.

A hydrocarbon product according to another embodiment of the present disclosure is produced using the above-described fermentation method.

A system for CO₂-free waste gas fermentation using microbial strains according to another embodiment of the present disclosure includes a bioreactor including microbial strains; a gas cylinder capable of injecting gaseous substrates into the bioreactor; and a liquid inlet capable of injecting methanol into the bioreactor, and the system is capable of converting, by injecting the gaseous substrates and the methanol into the bioreactor, the gaseous substrates and the methanol into hydrocarbon products through fermentation of the microbial strains.

The system for CO₂-free waste gas fermentation is capable of consuming and converting all CO₂ present in the bioreactor into hydrocarbon products.

The system for CO₂-free waste gas fermentation is capable of independently controlling one or more variables selected from the group consisting of an injection rate of the gaseous substrates, a pH inside the reactor and a mixing ratio of the gaseous substrates.

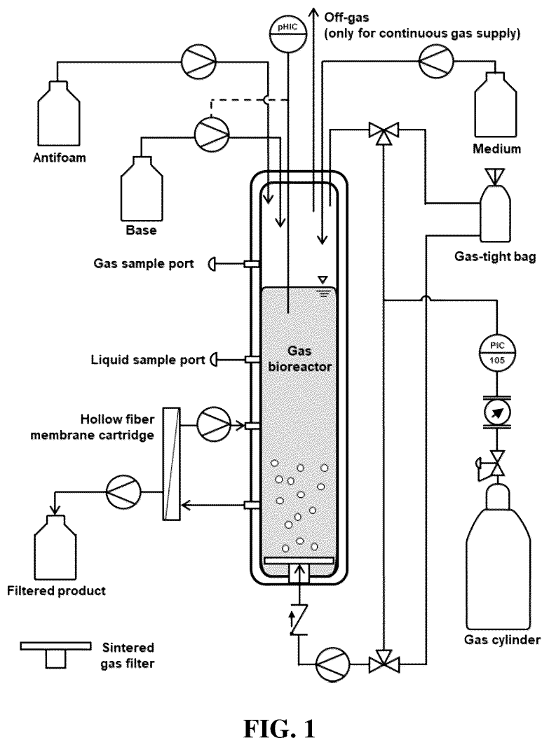

A mimetic diagram illustrating the system for CO₂-free waste gas fermentation using microbial strains is shown in FIG. 1.

When specifically describing the system for CO₂-free waste gas fermentation of the present disclosure referring to FIG. 1, a bioreactor in which microbial strains are injected to perform a fermentation step may be provided, and to the bioreactor, a gas cylinder capable of injecting gaseous substrates and a liquid inlet capable of injecting methanol are connected, and in addition thereto, a feed tank (Medium) capable of additionally supplying a medium and a base tank (Base) capable of controlling a pH inside the reactor may be connected.

Specifically, the bioreactor is a place where microbial strains consume gaseous substrates and methanol injected into the reactor and convert these into hydrocarbon products, and, as described above, consumption efficiency of the gaseous substrates by the microbial strains may be maximized by the injected methanol and H₂ and/or methanol additionally injected at a specific point, and ultimately, all the produced CO₂ is consumed and converted into hydrocarbon products. During this process, a pH inside the reactor is preferably maintained at 6.8 to 7.2 by the base tank, and 0.005 vvm to 0.010 vvm of the gaseous substrates may be injected by the gas cylinder.

### [Experimental Material Preparation and Method]

### Selection of Strain, Culture Method and Analysis Method

As an experimental strain, Eubacterium limosum KIST612 deposited at the Korean Collection for Type Cultures (KCTC) was used. E. limosum KIST612 was introduced to a 125 mL serum vial, and cultured under an anaerobic condition in a HEPES buffer basal medium (HBBM) at 37° C. Meanwhile, H₂/CO₂ (H₂:CO₂ [80:20]), N₂/CO₂ (N₂:CO₂ [80:20]) and synthetic syngas (CO:H₂:CO₂ [22:50:28]) were provided from “Dae Deok Gas Co., Ltd.” and used. As methods to measure cell concentration and gas, known methods were used, and for gas compound analyses, gas chromatography (ACME 6100, YoungIn Chromass, Korea) equipped with a thermal conductivity detector was used. Methanol and organic acids were analyzed at a flow rate of 0.6 mL-min¹ using high-performance liquid chromatography (YoungIn Chromass) equipped with a refractive index detector and an Aminex HPX-87H column (Bio-Rad, USA).

### Comparative Genomics and Sequence Analysis

Meanwhile, homologues proteins were searched using BLASTp, and the Genbank accession numbers used for the study were CP002273 (E. limosum KIST612), GCA_900562175 (Acetobacterium bakii), CP002987 (A. woodii), CP009170 (A. kivui), CP009687 (C. aceticum), CP020559 (C. formicaceticum), CP000232 (M. thermoacetica) and GCA_000007345 (Methanosarcina acetivorans), and visualization of nucleotide sequence identity was performed using BLASTn-based Kablammo.

### Gas Bioreactor Operation

A gas bioreactor (total volume 500 mL) designed in a laboratory was used with a working volume of 250 mL (FIG. 1). A sintered gas filter (pore diameter 10 μm to 16 μm, Daihan Scientific Co., Ltd., Korea) was installed at the bottom of the reactor, and the supply gas was separated to generate small bubbles. For the concentration of microbial cells and their accumulation in the bioreactor, a hollow fiber cartridge (CFP-2-E-3MA, GE Health Care, USA) was installed outside the reactor. Meanwhile, a fresh medium was continuously supplied to the bioreactor through controlling a dilution rate of the medium (exchange rate of medium), and the pH was adjusted to 7.0 using a controller (pHIC, consort R3610, Belgium). Meanwhile, the fermentation process of the microorganisms was performed under a known “simultaneous gas feeding and cell-recycling” (SGCR) mode. The SGCR mode is used to achieve high cell growth, and is capable of obtaining high reaction efficiency and substrate consumption rate without producing inhibitory products. In the growth phase of low cell concentration (<20 of OD₆₀₀), a mixed gas (CO:CO₂ [80:20]) was used as a gaseous substrate, and when the microbial cells in the bioreactor reached a steady state, syngas (CO:H₂:CO₂:Ar [11:25:14:50]) held in a gas-tight bag (SKC Inc., USA) was supplied. The supply of H₂ was controlled using a digital gas flow meter (Cole-Parmer, USA), and methanol was filter sterilized and injected into the bioreactor using a spiking mode.

### Calculation of Dissolved Gas Concentration

The concentration of dissolved gas in a liquid phase (C_{L,gas}) was calculated based on the Henry's constant. Specifically, the concentration of saturated gas (C*_{gas}) was calculated from the following Equation 1 and Equation 2, and the concentration of dissolved gas in a liquid phase (C_{L,gas}) was calculated from the following Equation 3 and C*_{gas}.

$$H ( T ) = {H}^{0} exp [ \frac{- {Δ}_{sol} H}{R} ( \frac{1}{T} - \frac{1}{{T}^{0}} ) ] \qquad [ Equation 1 ]$$

$${C}_{gas}^{*} = \frac{p}{{K}_{H}^{pc}} \qquad [ Equation 2 ]$$

$$\frac{{dC}_{L , gas}}{dt} = {k}_{L} a \cdot ( {C}_{gas}^{*} - {C}_{L , gas} ) - {q}_{gas} \cdot X \qquad [ Equation 3 ]$$

Equation 1 is the Van′t Hoff equation, and herein, H⁰ is the Henry's law constant (298.15K), ΔₛₒₗH is dissolution enthalpy, R is a molar gas constant, and T is a temperature (K).

Meanwhile, in Equation 2, K_{H}^{PC} represents Henry volatility defined through concentration (L·atm·mol⁻¹), and P means a partial pressure (atm). Particularly, the dissolved concentration of CO₂ is considered only as CO₂ [aq] without considering bicarbonate.

Meanwhile, in Equation 3, q_{gas} means a gas specific absorption rate (mol·gDCW⁻¹·h⁻¹), k_{L}a means a mass transfer coefficient, and X means a microbial cell concentration (g·L⁻¹). According to the calculation of C_{L,gas}, it was predicted that a portion of the gas would be restricted to the cell during the operation when deriving q_{gas}. Meanwhile, all other values were directly measured using each analysis equipment.

Meanwhile, productivity (P; g·L⁻¹·h⁻¹) was calculated according to the following Equation 4 in order to monitor the changes obtained by converting the main substrate consumption of CO, H₂/CO₂ and methanol.

$$P = \frac{D ( {C}_{t 1} - {e}^{D Δ t} \times {C}_{t 2} )}{1 - {e}^{D Δ E}} \qquad [ Equation 4 ]$$

In Equation 4, D represents a dilution rate (h⁻¹), and Ct means a product concentration (g·L⁻¹) at a specific time (t).

### Cumulative CO₂ Emission Modeling

A CO₂ emission model under the continuous gas supply was derived, and in order to derive the effect of additional substrates when converting the residual gases according to the parameters shown in the following Table 1, three operating conditions of the following Table 2 were used.

TABLE 1

| Parameter | Unit | Value |
|---|---|---|
| μₘₐₓ | h⁻¹ | 0.17 |
| D | h⁻¹ | 0.025 |
| K_{L}a_CO | h⁻¹ | 43.92 |
| K_{L}a_CO₂ | h⁻¹ | 11.16 |
| K_{L}a_H₂ | h⁻¹ | 66.96 |
| Y_{CO2/CO} | — | 0.50 |
| U_{CO2/H2} | — | 0.20 |
| U_{CO2/methanol} | — | 0.14 |
| qCO | mmol · gCDW⁻¹ h⁻¹ | 2.08 |
| qCO₂ | mmol · gCDW⁻¹ h⁻¹ | 1.16 |
| qH₂ | mmol · gCDW⁻¹ h⁻¹ | 5.92 |
| Xₒ | g · L⁻¹ | 0.14 |
| X_{steady state} | g · L⁻¹ | 9.80 |

(μₘₐₓ: maximum specific growth rate; D: dilution rate; k_{L}a: mass transfer coefficient; Y: yield coefficient; U: consumption ratio; q: specific uptake rate; X₀: initial cell concentration)

Specifically, condition A is a control operated without additional gaseous substrate supply, and condition B and condition C are conditions operated with additional supply of methanol, and methanol and hydrogen (H₂), respectively.

Meanwhile, the cell mass formation rate (dX/dt) in the simulation is shown in the following Equation 5.

$$\frac{dX}{dt} = μ \cdot X \qquad [ Equation 5 ]$$

Herein, the specific growth rate (μ) was calculated based on a separate vial test. Meanwhile, headspace gases of CO and H₂ (NCO and NH₂, respectively) were calculated according to the following Equations 6 and 7 based on the amount of each dissolved gas consumption in broth (V_{L}).

$$\frac{{dN}_{CO}}{dt} = {N}_{gas , in} \cdot {y}_{CO} - X \cdot {V}_{L} \cdot {q}_{CO} \qquad [ Equation 6 ]$$

$$\frac{{dN}_{{H}_{2}}}{dt} = {N}_{gas , in} \cdot {y}_{{H}_{2}} - X \cdot {V}_{L} \cdot {q}_{{H}_{2}} \qquad [ Equation 7 ]$$

Herein, N_{gas,in} means the number of moles of continuous gas supply to the bioreactor. Meanwhile, y_{co} and y_{H2} respectively mean mole fractions of CO and H₂ in the headspace. Meanwhile, CO₂ of the headspace (NCO₂) was simulated with CO, H₂ and methanol consumption data using the following Equation 8.

$$\frac{{dN}_{{CO}_{2}}}{dt} = {N}_{g , in} {y}_{{CO}_{2} , in} - X \cdot {V}_{L} \cdot ( {q}_{CO} \cdot {Y}_{\frac{{CO}_{2}}{CO}} - {q}_{{H}_{2}} \cdot {U}_{\frac{{CO}_{2}}{{H}_{2}}} - {q}_{MeOH} \cdot {U}_{\frac{{CO}_{2}}{MeOH}} ) \qquad [ Equation 8 ]$$

Herein, y_{CO2/CO} is a yield coefficient, and U_{CO2/H2} and U_{CO2/MeOH} are consumption ratios between CO/H₂ and CO/methanol in each experiment.

Meanwhile, the three operating conditions are shown in Table 2.

TABLE 2

Additional Feeding Size and Mode

| | H₂ | Methanol |
|---|---|---|
| A | — | — |
| B | — | 0.5 mol and spiked |
| C | 6 mol and continuous | 0.5 mol and spiked |

### [Experimental Results]

### Selection of Methylotrophic Acetogen Strain for Complete GTL (Gas to Liquids) Conversion

Based on the following Table 3, genetic similarity and culture characteristics of the acetogen strains were investigated to identify which strain would be an optimal candidate for the GTL reaction based on the characteristics of utilizing methanol and gas substrates.

TABLE 3

| | WGS | | | Optimal Growth | | |
|---|---|---|---|---|---|---|
| | (whole- | | | Conditions | | |
| | genome | Substrates | | Temp | | |
| Strains | sequencing) | H₂/CO₂ | CO | (° C.) | pH | Ref. |

Non-methylotrophic Acetogens

| | | | | | | |
|---|---|---|---|---|---|---|
| Acetoanaerobium | n.c.^{a} | + | − | 37 | 7.6-7.8 | 35 |
| noterae | | | | | | |
| Acetobacterium | n.c.^{b} | + | + | 30 | 7.5 | 36 |
| fimetarium | | | | | | |
| Clostridium | ◯ | + | + | 37 | 5.8-6.0 | 8 |
| autoethanogenum | | | | | | |
| C. carboxidivorans | ◯ | + | +^{a} | 37-40 | 5.0-7.0 | 37 |
| C. difficile | ◯ | + | − | 37 | 5.9 | 38 |
| C. drakei | ◯ | + | + | 30-37 | 5.4-7.5 | 37 |
| C. ljungdahlii | ◯ | + | + | 37 | 5.9 | 12 |
| C. scatologenes | ◯ | + | + | 37 | 7.0 | 37 |
| C. ultunense | n.c. | | − | 37 | 7.0 | 39 |
| | n.c.^{a} | | | | | |
| Oxobacter pfennigii | n.c.^{b} | + | + | 37 | 7.3 | 40 |

Methylotrophic Acetogens

| | | | | | | |
|---|---|---|---|---|---|---|
| A. bakii | ◯ | + | + | 25 | 7.3-7.7 | 36 |
| A. dehalogenans | n.c.^{a} | + | + | 25 | 7.3-7.7 | 41 |
| A. paludosum | n.c.^{b} | + | + | 20 | 6.8-7.0 | 36 |
| A. tundrae | n.c.^{b} | + | + | 20 | 7.0 | 42 |
| A. wieringae | n.c.^{a} | + | +^{a} | 30 | 7.6 | 43 |
| A. woodii | ◯ | + | +^{b} | 30 | 7.6 | 9 |
| Acetogenium kivui | ◯ | + | + | 60 | 6.8-7.0 | 11 |
| Butyribacterium | n.c.^{b} | + | + | 37-40 | 7.0-8.5 | 44 |
| methylotrophicum | | | | | | |
| C. aceticum | ◯ | + | +^{a} | 37 | 8.3 | 45 |
| C. formicaceticum | ◯ | | + | 37 | 8.0 | 46, 47 |
| C. magnum | n.c.^{a} | + | − | 30 | 7.0 | 48 |
| C. methoxybenzovorans | n.c.^{b} | + | − | 37 | 7.4 | 44, 46, 49 |
| Eubacterium aggregans | n.c.^{b} | + | − | 35 | 7.2 | 50 |
| E. limosum | ◯ | + | +^{a} | 37-39 | 7.2 | 10 |
| Moorella mulderi | n.c.^{b} | + | − | 65 | 7.0 | 51 |
| M. thermoacetica | ◯ | + | + | 55-60 | 6.8-6.9 | 52 |
| Sporomusa acidovorans | n.c.^{a} | + | − | 35 | 6.5-7.0 | 53 |
| S. ovata | n.c.^{a} | + | − | 34-39 | 6.3-7.0 | 13 |
| S. sphaeroides | —^{b} | + | − | 35-39 | 6.5-7.0 | 13 |

(n.c., not completed; ◯, completion; +, growth; − , no growth, ^{a}scaffold; ^{b}contig.)

Methanol utilization pathways in prokaryotes may be divided into two types of methanol dehydrogenase or methyltransferase depending on the enzymes involved. Most methylotrophic acetogen strains have methyltransferase that converts methanol into methyl-THF, an intermediate in a reductive acetyl-CoA pathway.

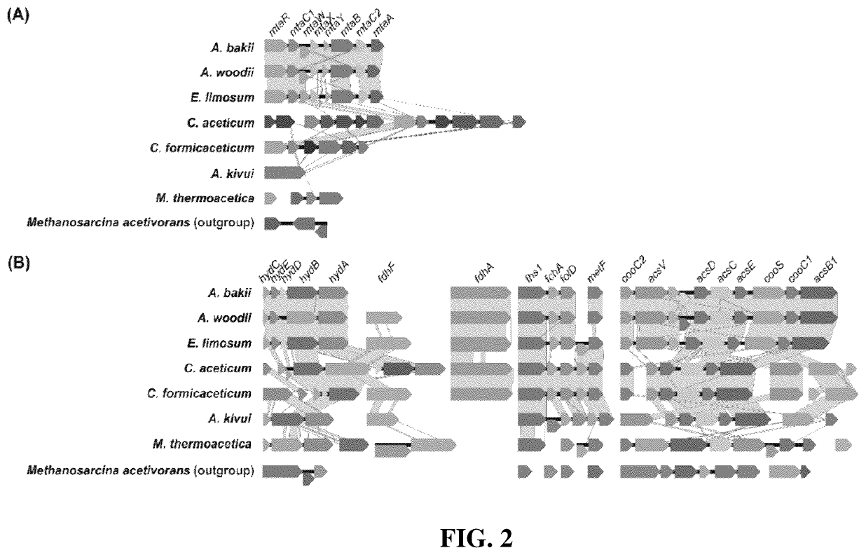

Accordingly, genome sequences of seven methylotrophic acetogen strains were analyzed. Although each strain was genetically distant from each other (ANI value <80), all of them were identified to have a methyltransferase gene cluster (FIG. 2A). In particular, it was identified that Acetobacterium and Eubacterium share the same mta operon having high sequence homology, and this mta operon is known to convert methanol into methyl-THF in A. woodii DSMZ 1030 and E. limosum ZL-II. Therefore, it may be assumed that the mta operon has undergone evolutionary mutation since an mta operon is taxonomically distinct as Acetobacterium or Eubacterium genus.

In addition, it may be deduced that C. aceticum has an mtb system (mtbA, mttWB and mtbC), A. kivui has only mtrH based on sequence identity, and the mtb/mtr system is for utilizing methanol in these strains. As a result, it may be inferred that methyltransferase is an enzyme that converts methanol into methyl-THF in most methylotrophic acetogen strains.

The inventors further investigated whether the methylotrophic condition similarly consumes gaseous substrates (for example: H₂/CO₂) in addition to CO. The inventors of the present disclosure identified that methanol accelerated the consumption rate of H₂ in E. limosum KIST612, however, how methanol affects H₂ metabolism has not yet been identified in previous studies. As it was assumed that there were clues within the reductive acetyl-CoA pathway, the reductive acetyl-CoA pathway of each strain was carefully compared (FIG. 2B).

Within the pathway, formate dehydrogenase (coded as fdh), formyltetrahydrofolate synthase (fhs), methenyltetrahydrofolate cyclohydrolase (fch), methylenetetrahydrofolate dehydrogenase (fol), methylenetetrahydrofolate reductase (met) and hydrogenase complex (hydABC) are enzymes on the methyl branch, and the CODH/ACS cluster corresponds to an enzyme complex on the carbonyl branch. Although the type and the order of each gene within the cluster differ between the strains, each gene was identified to maintain significant sequence homology for hydABC, fhs1, folD, metF and CODH cluster. Unique differences were also found in two genes, Fch and fdh. All the strains were identified to have fchA except M. thermoacetica that uses bifunctional methylenetetrahydrofolate dehydrogenase instead of methenyltetrahydrofolate cyclohydrolase. Therefore, the absence of fch does not affect the methanol utilization rate of M. thermoacetica.

Another characteristic is that most methylotrophic acetogen strains have both fdhF and fdhA. When detecting comparable genes in A. bakii based on sequence identity using BLASTp, fdhF was not able to be found, however, previous studies identified that fdhF is also present in A. bakii. In particular, fdhF was found in all methylotrophic strains, and the importance is presumed to be related to the interaction with hydrogenase. This implies that activation of H₂ metabolism through methanol is due to the action of fdhF when methanol oxidation progresses. In addition, through a proteomic analysis, it was identified that fdhF (ELI_0994) is a main type of fdh under a methanol condition of E. limosum KIST612. Accordingly, it may be identified that syngas fermentation by methanol provided in the present specification will be effective for all methylotrophic strains including strains lacking fdhA.

In addition, test strains were selected from among mesophiles and thermophiles since psychrophiles maintain a relatively low metabolic reaction rate. Meanwhile, strains with low CO tolerance were also excluded from the selection of test strains. Ultimately, candidate methylotrophic mesophilic/thermophilic strains of A. kivui, B. methyloptrophicum, C. aceticum, E. limosum and M. thermoacetica capable of consuming CO were selected.

Among these, relevant information on autotropic fermentation of M. thermoacetica was not able to be identified, and therefore, substrate consumption rate and product titre of the four strains except M. thermoacetica are shown in the following Table 4.

TABLE 4

| | Substrate | | | |
|---|---|---|---|---|
| | Consumption | Product | Gas | |
| | Rate | Titre | Composition | |
| Strain | (mmol · h⁻¹) | (g · L⁻¹) | (ratio as %) | Ref. |
| A. kivui | 0.021 as CO | 2.4 as acetate | CO:CO₂ | 61 |
| | | | (30:14) | |
| B. | — | 1.7 as acetate | CO:H₂:CO₂ | 46 |
| methylotrophicum | | 0.1 as butyrate | (20:30:30) | |
| C. aceticum | — | 15.7 as acetate | CO only | 62 |
| E. limosum | 10.6 as CO | 9.8 as acetate | CO:CO₂ | 32 |
| KIST612 | | 6.7 as butyrate | (80:20) | |

Referring to Table 4, it was identified that A. kivui had a very low CO consumption rate, and B. methylotrophicum had a low product titre compared to C. aceticum or E. limosum. Accordingly, with the reported high CO consumption and tolerance, E. limosum KIST612 was ultimately selected as the strain of the present disclosure.

Identification of H₂/Methanol Amount Required for Biphasic Mode of Syngas Fermentation and CO₂ Reduction

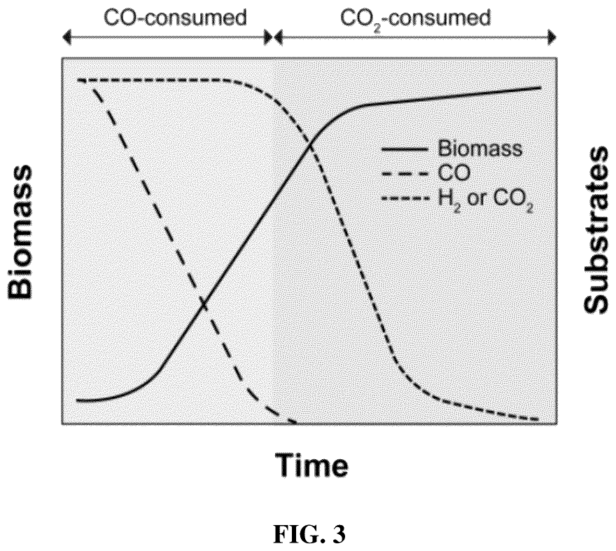

It was identified that, when CO was present in fermented syngas, activity of hydrogenase was inhibited by the CO, and H₂, an electron donor, was not properly metabolized during CO₂ reduction. Meanwhile, as a result of analyzing syngas fermentation data reported previously, it was identified that syngas fermentation has a two-phase reaction mode of a CO consumed stage and a CO₂ consumed stage (FIG. 3). Previous studies also identified that E. limosum KIST612 has this biphasic characteristic as well, and specifically, in the CO consumed stage, CO needs to be converted to CO₂, a prerequisite material for regulating a reductive acetyl-CoA pathway, to provide reducing power, and at this stage, it may be identified that net CO₂ production inevitably shows a positive value (+). Accordingly, as in the following Equation 9, 1 mol of CO theoretically produces 0.5 mol of CO₂ and 0.25 mol of acetate.

$$1 CO + 0.5 {H}_{2} O \to 0.25 acetate + 0.5 {CO}_{2} \qquad [ Equation 9 ]$$

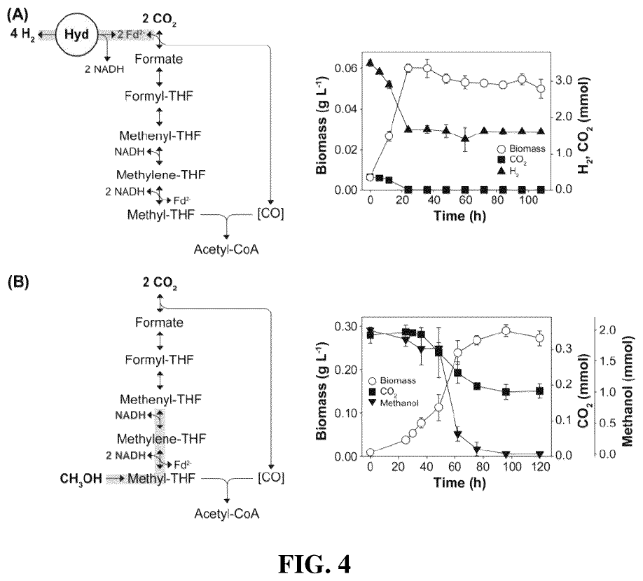

In previous studies, it was identified that 0.4 mol of CO₂ is produced per mol of CO consumed in a reaction of obtaining reduced ferredoxin from E. limosum KIST612. In the CO₂ consumed stage, reducing power needs to be secured through H₂ oxidation. In particular, it was identified that putative [FeFe]-hydrogenase (ELI_0847) annotated in the genome is present in E. limosum KIST612 (FIG. 4A), and it was identified that H₂ is consumed rapidly during the CO₂ consumed stage in E. limosum KIST612.

In addition, it was identified that, when only methanol was supplemented as an electron donor, electrons were obtained through a reverse reaction of the reductive acetyl-CoA pathway at this stage (FIG. 4B). In theory, 2 mol of H₂ or 1 mol of methanol may be used to reduce 0.5 mol of ferredoxin (oxidized form) and 1.5 mol of NAD⁺ through an oxidation reaction of H₂ or methanol, and phosphorous is used in a minimum amount required to utilize 1 mol of CO₂, however, these theoretical values may differ from actual data due to precise physiological dynamics during culture. Accordingly, as a result of calculating the required amounts of H₂ and methanol through the culture data obtained herein and the stoichiometric analysis, it was identified that 5 mol of H₂ or 7 mol of methanol are required when reducing 1 mol of CO₂ (FIG. 4).

### Monitoring Gaseous Substrate Consumption Depending on Presence or Absence of Methanol Supply During Syngas Fermentation

In order to evaluate the effect of methanol on the consumption of syngas components, E. limosum KIST612 was cultured under a syngas (CO:H₂:CO₂ [22:50:28]) condition regardless of the presence or absence of methanol. According to the stoichiometric analysis for CO₂ reduction, the methanol concentration was set to 100 mM at a working volume of 60 mL (corresponding to 6 mmol), and all CO₂ remaining from the net production and CO₂ that had been present in the original syngas were removed.

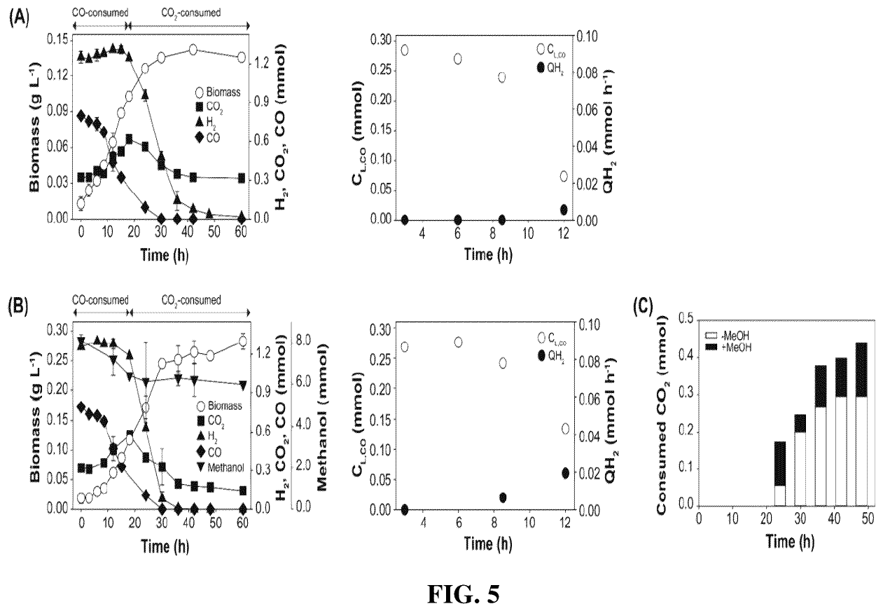

Based on the reasoning that methanol would accelerate H₂ consumption, methanol was supplied at the beginning of the reaction. Whereas FIGS. 5A and 5B identified that methanol supply was only effective in the stage of consuming CO₂ while simultaneously consuming H₂, there were no significant differences in the substrate consumption in the CO consumed stage (that is, stage of utilizing only CO as gaseous substrate).

Meanwhile, it may be identified that supplying methanol further accelerates cell growth and accelerates H₂ and CO₂ consumption, and as a result, residual CO₂ is reduced. Specifically, it was identified that, after 60 hours, the microbial cell concentration was approximately two times higher under the methanol supplying culture condition compared to under the culture condition in which methanol was not supplied. Alternatively, the growth rate during the CO consumed stage was not stimulated by the methanol addition, and this indicates that the enzyme for utilizing methanol is inhibited by the presence of the CO-like enzyme during H₂ metabolism.

Meanwhile, under the methanol supplying condition, H₂ consumption started approximately after 10 hours after inoculation, and it may be identified that this is significantly faster compared to when methanol was not supplied.

Specifically, referring to the right panel of FIG. 5A and the right panel of FIG. 5B, the measured partial pressure of CO indicates that, when H₂ consumption begin in both cases, the concentration of dissolved CO is greater than 0.25 mmol·L⁻¹. This may be due to mass transfer kinetics of the gaseous substances in addition to the actual CO concentration, which is due to the fact that the dissolved form has a significantly lower concentration compared to that calculated from the CO partial pressure corresponding to an inhibitory concentration of hydrogenase or any enzyme involved in H₂ metabolism. Meanwhile, the amounts of CO₂ consumed in both cases and the difference may be identified in FIG. 5C.

### Operation of Gas Bioreactor for Complete GTL (Gas to Liquid) Reaction

The following Table 5 shows production rates of CO₂ during syngas fermentation for several representative strains in various bioreactor types.

TABLE 5

| | Reactor Type | CO₂ Production | |
|---|---|---|---|
| | and Syngas | Rate | |
| Strain | Composition | (mmol · h⁻¹) | Ref |
| E. limosum | B-BCR | 4.97 | 32 |
| KIST612 | CO:CO₂ (80:20) | | |
| | C-BCR | 3.51 | 32 |
| | CO:CO₂ (80:20) | | |
| | M-BCR | 7.60 | 32 |
| | CO:CO₂ (80:20) | | |
| C. carboxidivorans | BSTR | 2.28 | 68 |
| | CO:CO₂ (80:20) | | |
| C. ljungdahlii | CSTR, BCR | 11.76 | 69 |
| PETC | CO:H₂:CO₂ (60:35:5) | | |
| C. ljungdahlii | CSTR, BCR | 6.60 | 69 |
| ERI-2 | CO:H₂:CO₂ (60:35:5) | | |
| C. autoethanogenum | CSTR, BCR | 12.42 | 69 |
| JA1-1 | CO:H₂:CO₂ (60:35:5) | | |

(BCR, bubble column reactor; B-, batch mode; C-, continuous mode; M-, mixed feeding mode; STR, stirred tank reactor; CSTR, continuous stirred tank reactor)

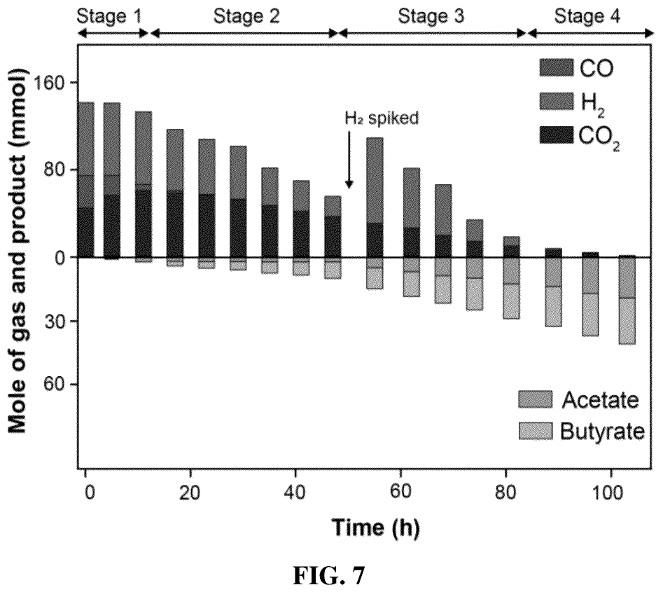

According to the research results, it was identified that CO₂ was produced in most of the syngas fermentation cases. Accordingly, newly produced CO₂ needs to be included in the gas that needs to be converted to a target product. During the bioreactor operation, once the cell concentration had stabilized at approximately 4.6 g·L⁻¹ (corresponding to 17.04 of OD₆₀₀), the gas supply mode was changed from direct supply of mixed gas (CO:CO₂:Ar [180:15:5]) through a gas cylinder to synthetic syngas (CO:H₂:CO₂ [22:50:28]) through a gas-tight bag. The methanol was injected into the bioreactor up to a maximum of 400 mM (corresponding to 100 mmol at working volume of 250 ml), which theoretically reacts with 100 mmol of CO₂ (FIG. 6A). Meanwhile, FIG. 7 shows the mole fractions of gas compounds and products in the gas and liquid phases over time.

After changing the gas supply mode to a gas-tight bag, 1.93 mmol·h⁻¹ of CO was consumed over 11 hours (Stage 1), and according to the gas chromatography analysis, the actual C_{L,co} was recorded at an undetectable concentration, and it may be identified that consumption of H₂, CO₂ and methanol has begun while CO is still present in the gas phase. Meanwhile, it may be identified that the CO₂ consumption rate exceeds the production rate for 14 hours as soon as the H₂ consumption begins (FIGS. 6B and C). In addition, it was identified that, in the stage 2, 45% of CO₂ was consumed in the gas-tight bag including CO₂ originally present in the supplied syngas and CO₂ produced during the CO oxidation process of the stage 1.

In order to more efficiently upcycle the remaining CO₂, a method of simultaneously adding H₂ and methanol to the reactor was used, and as a result, it was identified that 90% or more of the total CO₂ was consumed in 85 hours (stage 3).

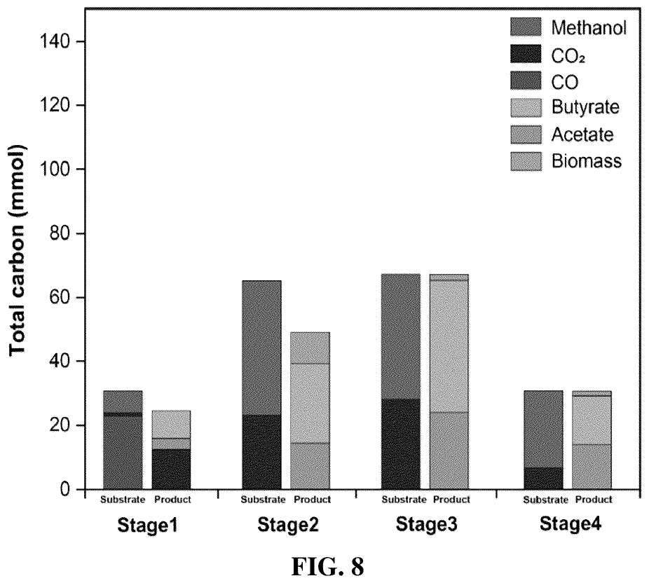

Accordingly, it was identified that methanol, a liquid chemical, is readily supplied and controlled in the concentration compared to H₂, making it a more preferable option for efficient consumption of remaining CO₂. Meanwhile, in the stage 4, methanol was additionally supplied for complete conversion, and remaining CO₂ was completely consumed. Through the fermentation process, acetate (10.40 mmol) and butyrate (5.62 mmol), two chemical liquid types, were produced (FIG. 6D), and successful conversion of the syngas to a liquid was identified through FIG. 8 based on the total number of carbons (including methanol, mmol) supplied from the substrate, and as a result, carbon balances in the stage 1 and the stage 2 were identified to be 80% and 75%, respectively. Herein, it is possible that each carbon balance is low due to changes in the main substrate. However, the syngas was successfully converted into a liquid chemical in the stabilized fermentation stage, and 99% of carbon balance was obtained in the stage 3 and the stage 4 (FIG. 8).

Overall, whereas the stage 1 and the stage 2 were CO consumed and CO₂ consumed stages and capable of identifying a two-phase mode of syngas fermentation, the stage 3 and the stage 4 were identified to promote the consumption of remaining CO₂ in order to accomplish complete GTL (gas to liquids) conversion.

### Modeling of CO₂ Emission Behavior of GTL (Gas to Liquids) Process

A modeling experiment to predict CO₂ emission behavior depending on the elapsed time of the complete GTL reaction was conducted. In particular, since the composition and the ratio of syngas vary depending on the supply material type and the reaction condition for gas production, having a supply gas with reliable change and composition for modeling is required. Accordingly, although the supply gas has a different composition ratio, it was assumed that three types of gases (CO, H₂ and CO₂) exist as only components of the supply gas (Table 2).

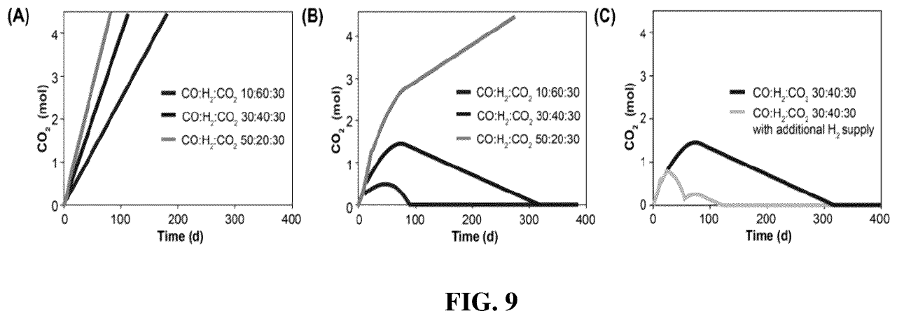

Meanwhile, FIG. 9 shows the results of predicting CO₂ emission behavior depending on the supplementation of supplied methanol, and it may be identified that, in all cases, a positive net CO₂ emission is obtained when methanol is not supplied (FIG. 9A). In addition, when methanol was additionally added, it was identified that, as a result of predicting the whole GTL scenario having the syngas CO:H₂:CO₂ ratios of 10:60:30 and 30:40:30 (FIG. 9B), the CO₂ consumption rate predicted in these two cases was higher than the CO₂ emission, and in the scenario (10%) with lower CO, the time taken until the GTL reaction was completed was predicted to be shorter. In particular, in the maximum CO % (50:20:30), the CO₂ emission did not decrease over time. These results may mean that the CO:H₂ ratio in the syngas composition is an important factor affecting the complete GTL reaction.

In addition, additional supply of H₂ was predicted to accelerate the GTL reaction as it meets the criterion of no CO₂ emission compared to an intermediate ratio of CO and H₂ (that is, 30:40:30) (FIG. 9C). Ultimately, the simulation results mean that the entire gas components are able to be successfully converted into a liquid phase without CO₂ emission. Meanwhile, what is important is that, by analyzing the gas composition and ratio of syngas supplied before operating the reactor, the amount and the timing of methanol or H₂ supply required to accomplish the condition of no CO₂ emission may be identified.

Hereinbefore, preferred embodiments of the present disclosure have been described in detail, however, the scope of a right of the present disclosure is not limited thereto, and various modified and improved forms made by those skilled in the art using the basic concept of the present disclosure defined in the claims also fall within the scope of a right of the present disclosure.

1. A method for CO₂-free waste gas fermentation using microbial strains, the method comprising:
1) injecting microbial strains into a bioreactor; and
2) injecting gaseous substrates and methanol into the bioreactor, and converting the gaseous substrates and the methanol into hydrocarbon products through fermentation of the microbial strains,
wherein all CO₂ present inside the bioreactor is consumed and converted into hydrocarbon products.

2. The method of claim 1, wherein the gaseous substrate includes one or more gases selected from the group consisting of CO, H₂ and CO₂.

3. The method of claim 1, wherein the hydrocarbon product is selected from the group consisting of acetate, butyrate, ethanol, propanol, butanol, 2,3-butanediol, propionate, caproate, propylene, butadiene, isobutylene, ethylene and mixtures thereof.

4. The method of claim 1, wherein the microbial strain is an acetogen strain, and the acetogen strain is one or more strains selected from the group consisting of Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, Moorella thermoacetica and Eubacterium callanderi.

5. The method of claim 1, wherein the methanol increases a consumption rate of the gaseous substrates by the microbial strains.

6. The method of claim 1, wherein a pH condition inside the bioreactor is from 6.8 to 7.2.

7. A hydrocarbon product produced using the method of claim 1.

8. A system for CO₂-free waste gas fermentation using microbial strains, the system comprising:
a bioreactor including microbial strains;
a gas cylinder capable of injecting gaseous substrates into the bioreactor; and
a liquid inlet capable of injecting methanol into the bioreactor,
wherein the gaseous substrates and the methanol are injected into the bioreactor, and the gaseous substrates and the methanol are converted into hydrocarbon products through fermentation of the microbial strains.

9. The system of claim 8, which is capable of consuming and converting all CO₂ present in the bioreactor into hydrocarbon products.

10. The system of claim 8, which is capable of independently controlling one or more variables selected from the group consisting of an injection rate of the gaseous substrates, a pH inside the reactor and a mixing ratio of the gaseous substrates.
